(19) United States
(12) Patent Application Publication
(10) Pub. No.: US 2015/0157309 A1
(43) Pub. Date: Jun. 11, 2015

(54) DEVICES, APPARATUSES, AND METHODS FOR TISSUE SUSPENSION

(71) Applicant: SCOTT & WHITE HEALTHCARE, TEMPLE, TX (US)

(72) Inventor: Erin T. Bird, Salado, TX (US)

(73) Assignee: Scott & White Healthcare, Temple, TX (US)

(21) Appl. No.: 14/381,925

(22) PCT Filed: Feb. 25, 2013
(86) PCT No.: PCT/US13/27652

§ 371 Date: Aug. 28, 2014

Related U.S. Application Data
(60) Provisional application No. 61/604,372, filed on Feb. 28, 2012.

(51) Int. Cl. A61B 17/04

(52) U.S. Cl. CPC A61B 17/0401; A61B 2017/00004

(57) ABSTRACT

Tissue suspension (e.g., bladder neck, urethral, etc.) devices, apparatuses, kits, and methods, such as those, for example, configured to suspend tissue at a target area (e.g., near a urethra, within a vagina, etc.) without tissue at the target area having been incised prior to using the devices, apparatuses, kits and/or methods.

### CROSS-REFERENCE TO RELATED APPLICATIONS

This application claims priority to U.S. Provisional Patent Application No. 61/604,372, filed Feb. 28, 2012, which is incorporated by reference in its entirety.

### BACKGROUND

1. Field of the Invention

The present invention relates generally to tissue suspension (e.g., bladder neck, urethral, etc.), and more particularly, but not by way of limitation, to devices, apparatuses, and methods for inserting a fixation device to suspend tissue without tissue at a target area (e.g., near a urethra, within a vagina, etc.) having been incised prior to insertion of the fixation device.

2. Description of Related Art

Examples of devices, apparatuses and/or methods for tissue suspension are disclosed in U.S. Pat. No. 5,591,163, U.S. Pat. No. 6,334,446, U.S. Pat. No. 6,506,190, U.S. Pat. No. 7,217,238, U.S. Pat. No. 8,047,983, and U.S. Pat. No. 8,057,383.

### SUMMARY

This disclosure includes embodiments of devices, apparatuses, kits, and methods for suspending tissue.

Some embodiments of the present devices comprise: a tip configured to penetrate tissue of a patient at a target area, a non-absorbable suture having a first end coupled to the tip and a second end, the non-absorbable suture comprising a plurality of barbs configured to resist movement of the fixation device within the tissue of the target area, an absorbable suture coupled to the second end of the non-absorbable suture, and a stay tab coupled to the absorbable suture, the stay tab configured to substantially prevent at least some tissue from moving away from the tip if the fixation device is implanted in the tissue of the target area. In some embodiments, the stay tab can further be configured to limit the depth to which at least a portion of the fixation device can enter the tissue of the target area. The fixation device can have a length of, for example, between 0.5 centimeters and 3 centimeters. In some embodiments, the tip of the fixation device can be configured to penetrate the tissue at the target area without the tissue having been incised prior to penetration of the tip. The tip can comprise a first material, the non-absorbable suture can comprise a second material, the absorbable suture can comprise a third material, and the stay tab can comprise a fourth material. In some embodiments, the first material can be the same as the second material. The first material can be any biocompatible material (e.g., silicone) configured with sufficient rigidity to penetrate tissue at the target area without the tissue having been incised prior to penetration of the tip. In some embodiments, the absorbable suture can comprise a plurality of barbs configured to resist movement of the fixation device within the tissue of the target area. The absorbable suture can further be configured to be absorbed by a patient's body to decouple the stay tab from the non-absorbable suture. The absorbable suture can also be configured to be absorbed such that the stay tab will not de-couple from the non-absorbable suture in less than two weeks and/or between two to four weeks.

Some embodiments of the present apparatuses comprise: an apparatus to deploy a fixation device comprising a shaft having a first end and a second end; a head coupled to the first end of the shaft, the head comprising a plurality of guides disposed within the head, where each of the plurality of guides is (i) configured to at least partially accommodate a fixation device, (ii) configured to extend from the head into a deployed configuration, (iii) configured to retract into the head into a retracted configuration; and a handle extending from the second end of the shaft, the handle comprising a lever configured to be actuated to extend at least one of the plurality of guides from the head. In some embodiments, each of the plurality of guides is further biased toward the retracted configuration. In some embodiments, the handle can comprise a switch configured to release at least a portion of at least one of the plurality of fixation devices from the at least one of the plurality of guides when the at least one fixation device is at least partially within the tissue at the target area.

In some embodiments, a plurality of fixation devices can be at least partially disposed within the plurality of guides. In some embodiments, the tip of the plurality of fixation devices can extend from the head when the plurality of guides are in the retracted configuration. In other embodiments, the plurality of fixation devices can be disposed within the head such that a surface of the head from which the plurality of guides extends is substantially smooth when the plurality of guides are in the retracted configuration. In some embodiments, at least one of the plurality of guides can enter the tissue at the target area when the lever is actuated. The plurality of fixation devices can be configured such that when the plurality of guides retract from the target area into the retracted configuration, the plurality of fixation devices remain at least partially within the tissue at the target area. The plurality of guides can comprise any biocompatible material configured with sufficient rigidity to enter the target area (e.g., a biocompatible metal, a biocompatible plastic, etc.).

In some embodiments, the plurality of guides can be disposed within the head in at least one row. In some embodiments, the head can a length between 0.5 centimeters and 2 centimeters. In some embodiments, the head is coupled to the first end of the shaft at an angle between 150 degrees and 180 degrees. In other embodiments, the head is coupled to the first end of the shaft at an angle configured to permit the head to be positioned substantially parallel to the tissue at the target area. In some embodiments, the head can further comprise at least one spring that can be (i) in a compressed configuration when the plurality of guides are in the retracted configuration, (ii) coupled to the lever such that the lever will release the at least one spring from the compressed configuration if the lever is actuated, and (iii) configured to apply a force, if released, to at least one of the plurality of guides such that the at least one guide extends from the head into the deployed configuration. In some embodiments, the at least one spring can be configured to stop applying the force to the at least one guide when the at least one guide is in the deployed configuration such that the at least one guide can retract into the retracted configuration.

Some embodiments of the present kits comprise: one or more of the present apparatuses, devices, and/or components thereof. Some embodiments further comprise instructions and/or other descriptions of methods (e.g., in a hard copy and/or computer readable) for using the one or more apparatuses and/or devices.

Some embodiments of the present methods comprise (i) inserting into a vagina of a patient an apparatus comprising a plurality of fixation devices disposed at least partially within a plurality of guides and (ii) deploying the apparatus such that at least one fixation device penetrates the tissue at the target area without the at least one fixation device being deployed into the bone or such that at least one fixation device penetrates the tissue at the target area without the tissue having been incised prior to penetration of the at least one fixation device. Some embodiments further comprise: (iii) positioning the apparatus substantially parallel to tissue of the patient at a target area near a urethra, (iv) releasing the at least one fixation device at least partially within the tissue at the target area with a switch, (v) allowing the at least one of guide to retract into a retracted configuration, (vi) repeating steps (iii)-(v) until a sufficient number of the plurality of fixation devices are at least partially within the tissue at the target area to suspend the urethra, and/or (vii) permitting the absorbable suture of at least one fixation device to be absorbed such that at least one stay tab can decouple from the at least one fixation device. Some embodiments further comprise successively deploying at least one fixation device on either side of the urethra. Some embodiments further comprise positioning an apparatus such that at least one of the plurality of guides is positioned on either side of the urethra such that the apparatus can deploy at least one of a plurality of fixation devices on either side of the urethra without repositioning the apparatus.

Any embodiment of any of the present devices and apparatuses can consist of or consist essentially of—rather than comprise/include/contain/have—any of the described elements and/or features. The same is true of the present methods. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The embodiments of the present tissue suspension devices and apparatuses and their components shown in FIGS. 5-13 are drawn to scale for at least the depicted embodiment.

FIG. 1 depicts a side view of one embodiment of a tissue suspension fixation device.

FIG. 2 depicts a perspective view of one embodiment of an apparatus to deploy one or more fixation devices to suspend tissue.

FIG. 3 depicts a side view of one embodiment of the present tissue suspension devices and apparatuses positioned at a target area (e.g., within a vagina).

FIG. 4 depicts one example of possible target areas to deploy one or more of the plurality of fixation devices (e.g., within the vagina).

FIG. 5 depicts a perspective view of one embodiment of a tip of the present fixation devices.

FIG. 6 depicts a front view of the tip of FIG. 5.

FIG. 7 depicts side view of the tip of FIG. 5.

FIG. 8 depicts a cross-sectional front view of the tip of FIG. 5.

FIG. 9 depicts a bottom view of the tip of FIG. 5.

FIG. 10 depicts a top view of the tip of FIG. 5.

FIG. 11 depicts a perspective view of one embodiment of a stay tab of the present fixation devices.

FIG. 12 depicts a top view of the stay tab of FIG. 11.

FIG. 13 depicts a side view of the stay tab of FIG. 11.

### DESCRIPTION OF ILLUSTRATIVE EMBODIMENTS

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, devices or apparatuses for tissue suspension, or a component of the devices and apparatuses, that is configured in a certain way is configured in at least that way, but it can also be configured in other ways than those specifically described.

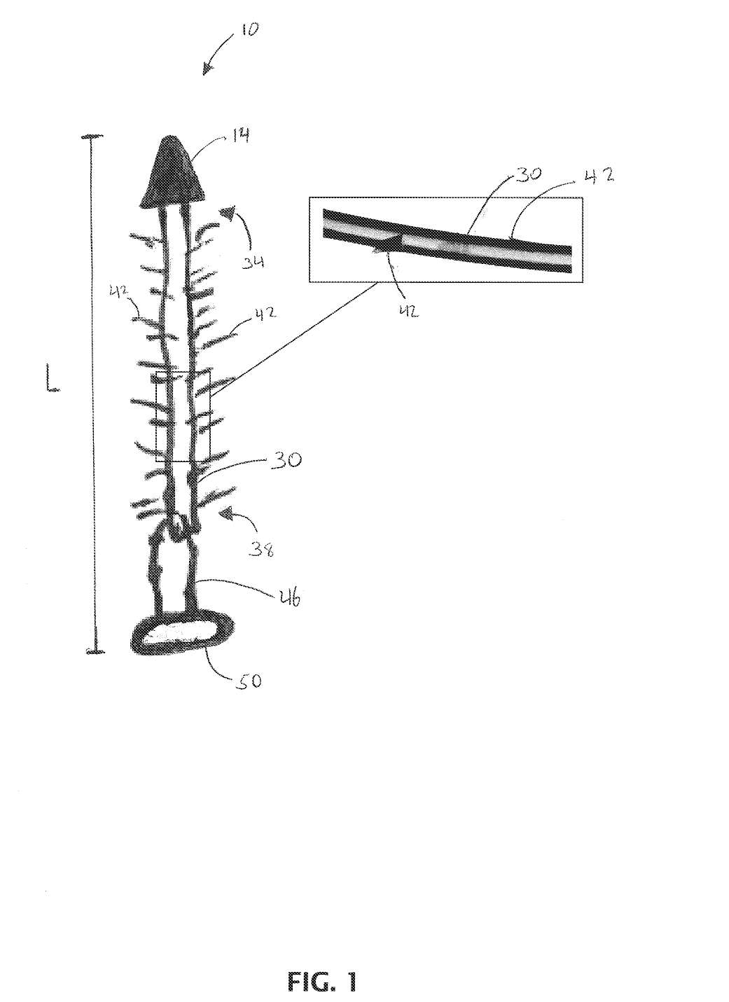

Referring now to the drawings, and more particularly to FIGS. 1-4, shown therein and designated by the reference numeral 10 is one embodiment of the present tissue suspension devices. In the embodiment shown, fixation device 10 comprises a length L that can, for example, be between 0.2 centimeters and 5 centimeters. In some embodiments, fixation device 10 can comprise a length L that can be between 0.5 centimeters and 3 centimeters. In some embodiments, fixation device 10 can comprise a length L that can be between 1 centimeters and 2 centimeters. In other embodiments, length L can be less than 0.5 centimeters. In other embodiments, length L can be greater than 5 centimeters. In other embodiments, fixation device 10 can have any suitable length L configured to permit suspension of tissue in a target area (e.g., as described in this disclosure). In some embodiments, fixation device(s) 10 can comprise and/or be coated with one or more antibiotics and/or other medications (e.g., pain medication, etc.).

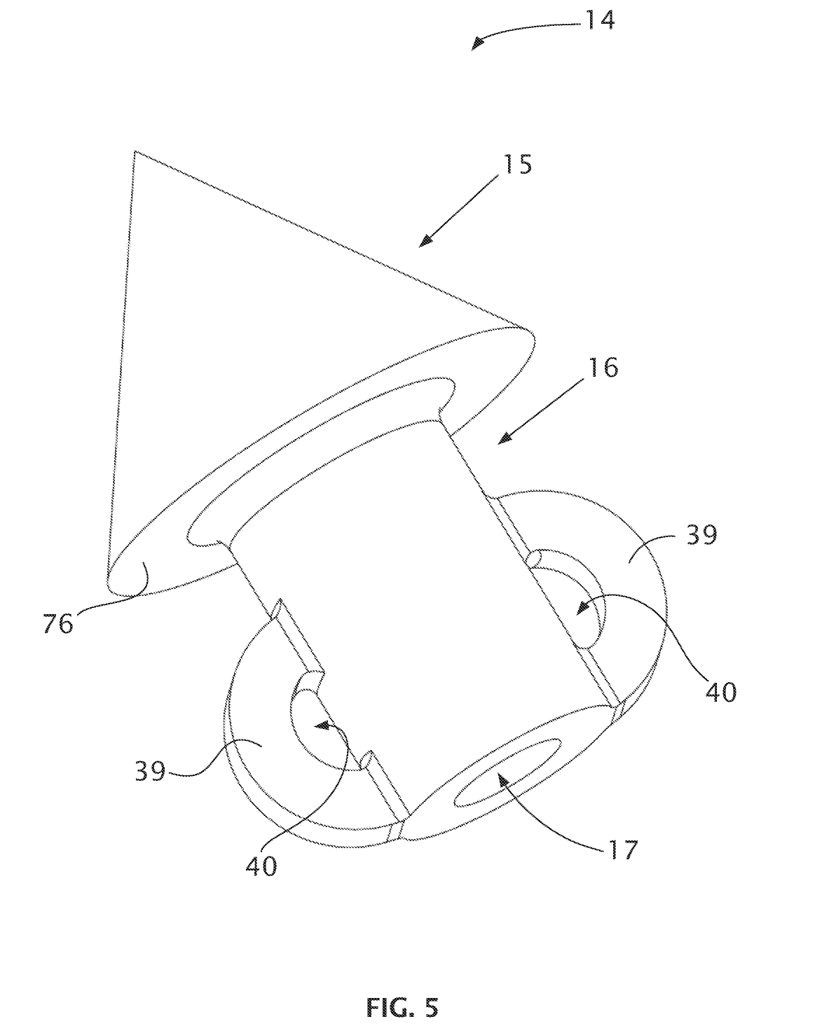

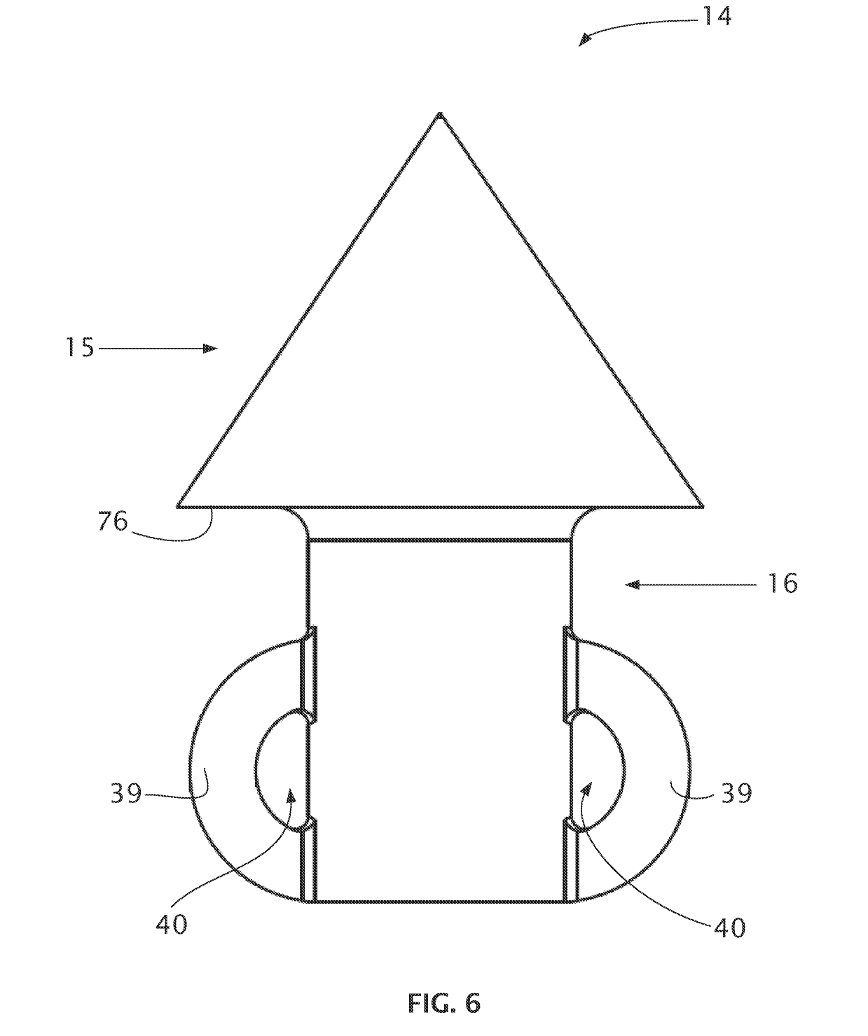

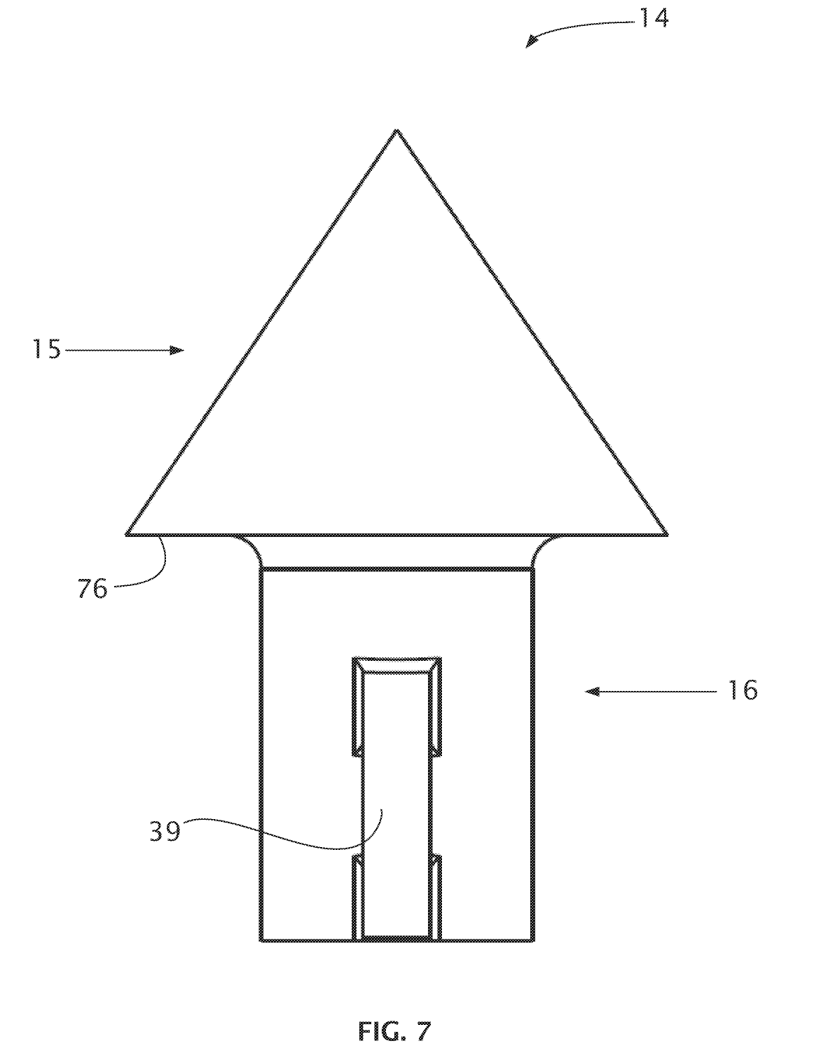

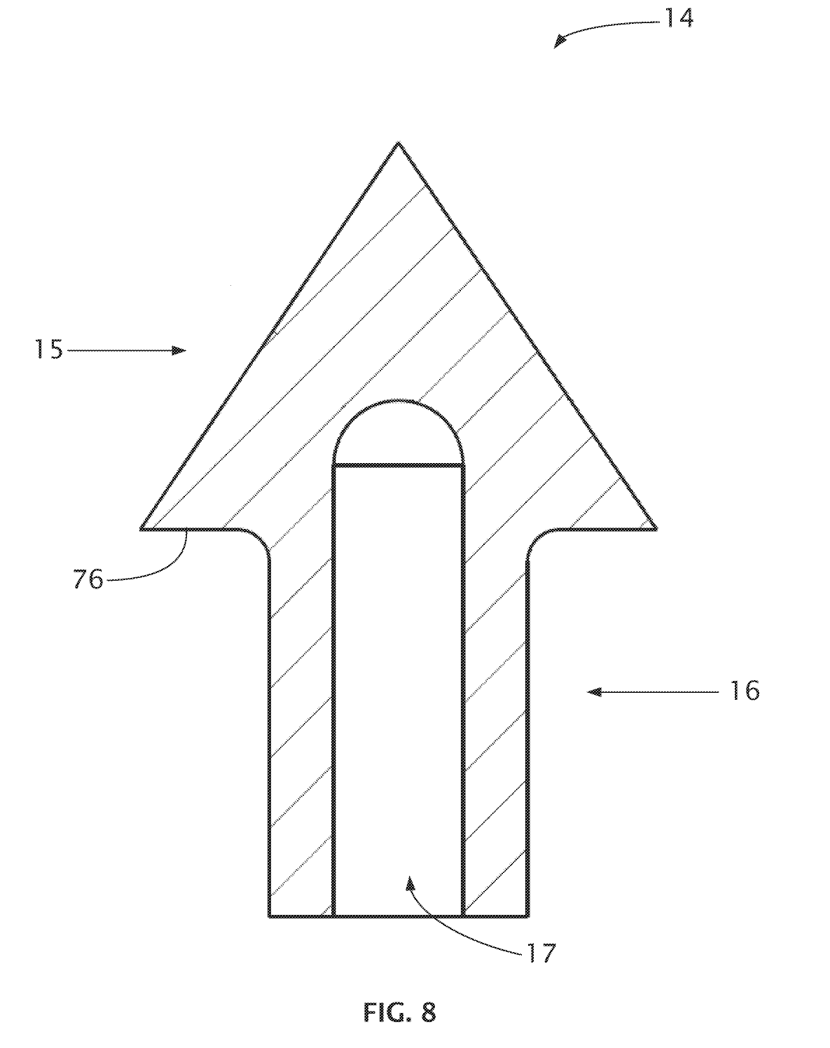

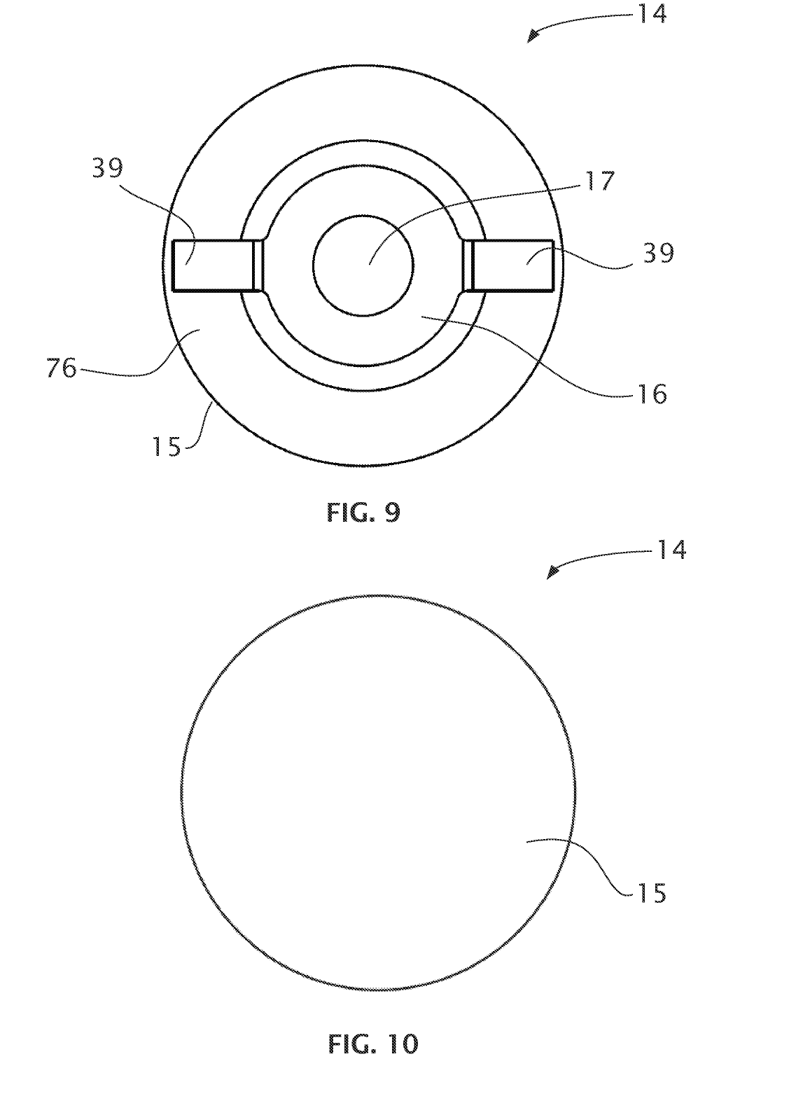

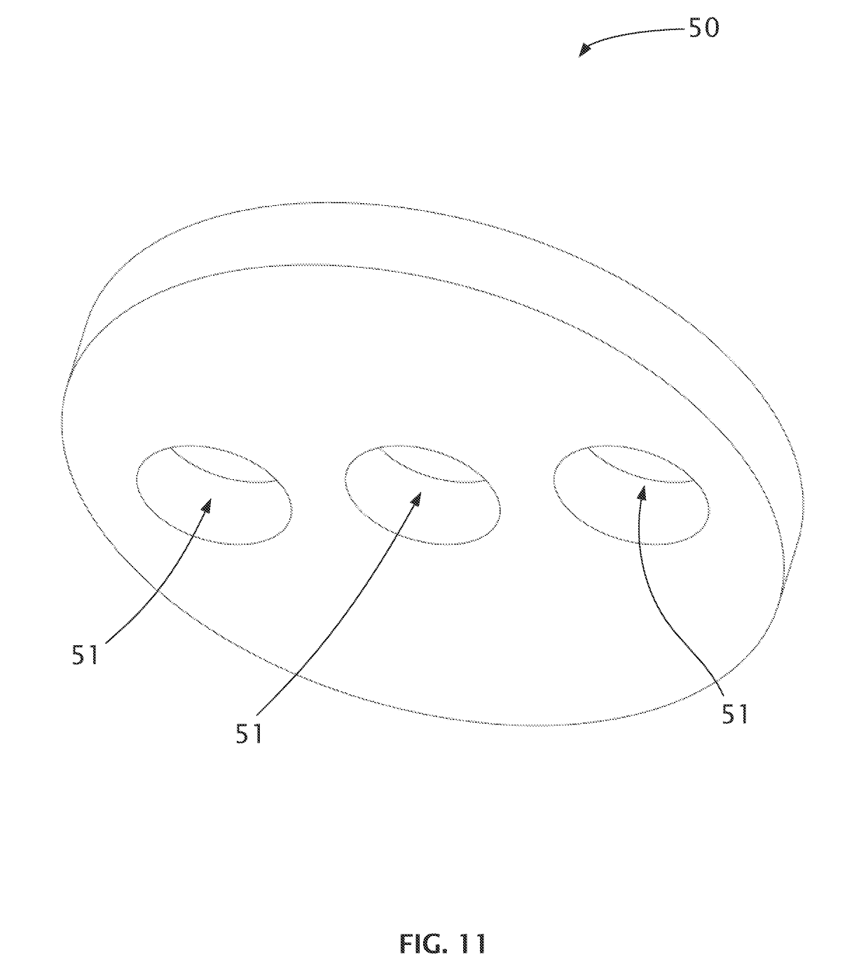

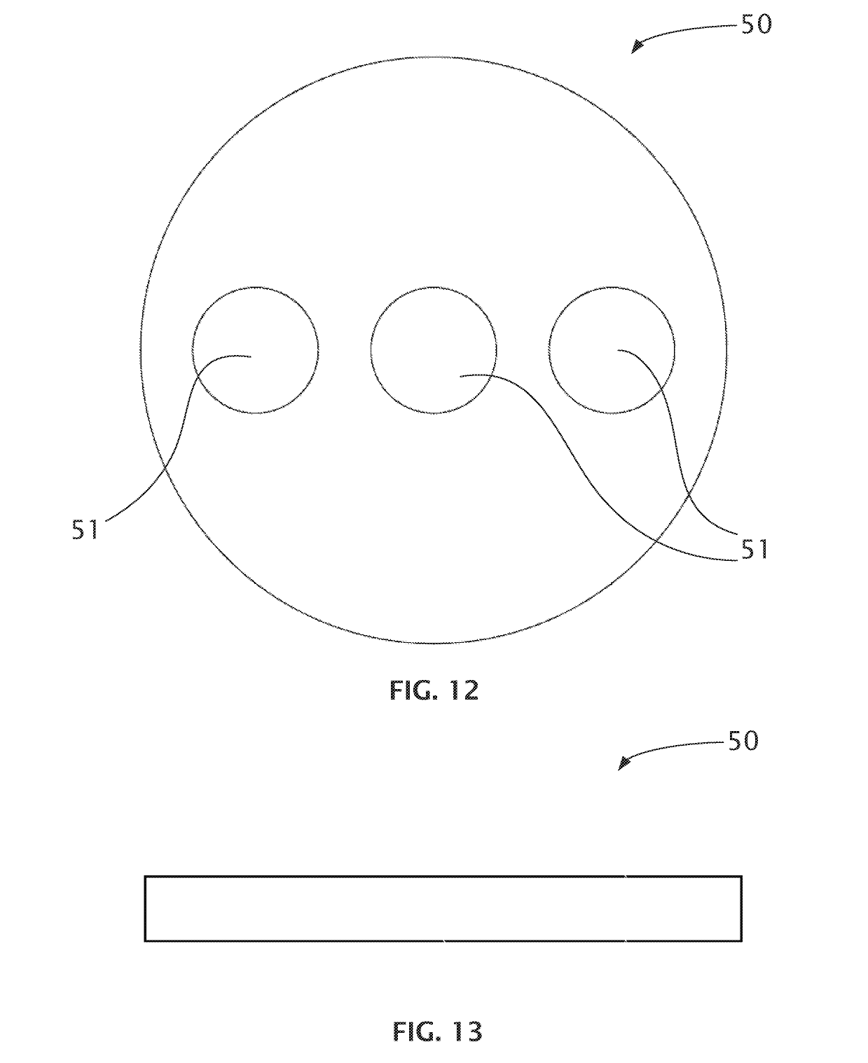

In the embodiment shown in FIGS. 1-3 and 5-10, for example, fixation device 10 comprises a tip 14. As depicted in the embodiments shown in FIGS. 5-8, tip 14 can comprise upper portion 15 and lower portion 16. In some embodiments, upper portion 15 and lower portion 16 are coupled to one another (e.g., via welding, adhesive, and the like); and in other embodiments, upper portion 15 and lower portion 16 are unitary (e.g., formed of a single piece of material). In the embodiment shown, upper portion 15 is substantially conical. However, in other embodiments, upper portion 15 can be any suitable shape configured to penetrate a target area. Also in the embodiment shown, lower portion 16 is substantially cylindrical. In the embodiment shown, lower portion 16 comprises bore 17 extending at least partially through lower portion 16. In the embodiment shown, bore 17 is substantially cylindrical. Further, in the embodiment shown, bore 17 extends through all of lower portion 16 and extends into a portion of upper portion 15 of tip 14.

Tip 14 can comprise any suitable length depending on, for example, characteristics of a target area, a desired depth of penetration, and the like. For example, tip 14 can comprise a length of 0.1 millimeter to 1 centimeter (e.g., 6 millimeters). In some embodiments, upper portion 15 and lower portion 16 comprise the same length (e.g., 3 millimeters); and in other embodiments, upper portion 15 and lower portion 16 can comprise different lengths. Tip 14 (e.g., upper portion 15 and lower portion 16) can also comprise any suitable diameter, such as, for example, a diameter of 0.1 millimeter to 1 centimeter. For example, upper portion 15 can comprise a diameter of 4 millimeters, and lower portion 16 can comprise a diameter of 2 millimeters. Similarly, bore 17 can comprise any suitable diameter, such as, for example, a diameter of 0.1 millimeters to 1 centimeter (e.g., 1 millimeter). As described above, bore 17 can extend through tip 14 for any suitable distance, such as, for example, a distance of 0.1 millimeters to 1 centimeter (e.g., 4 millimeter).

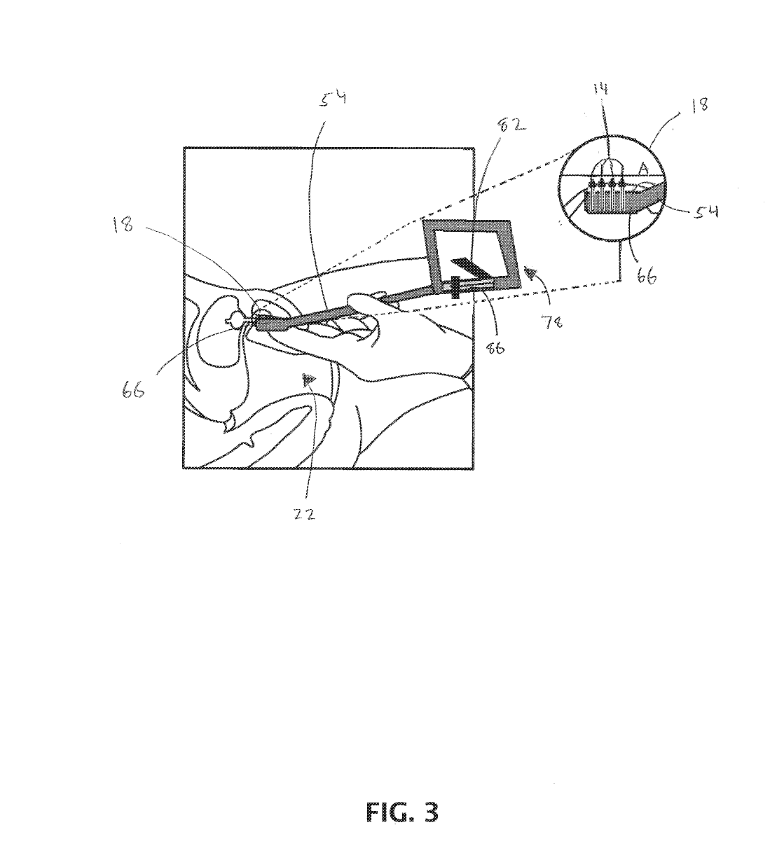

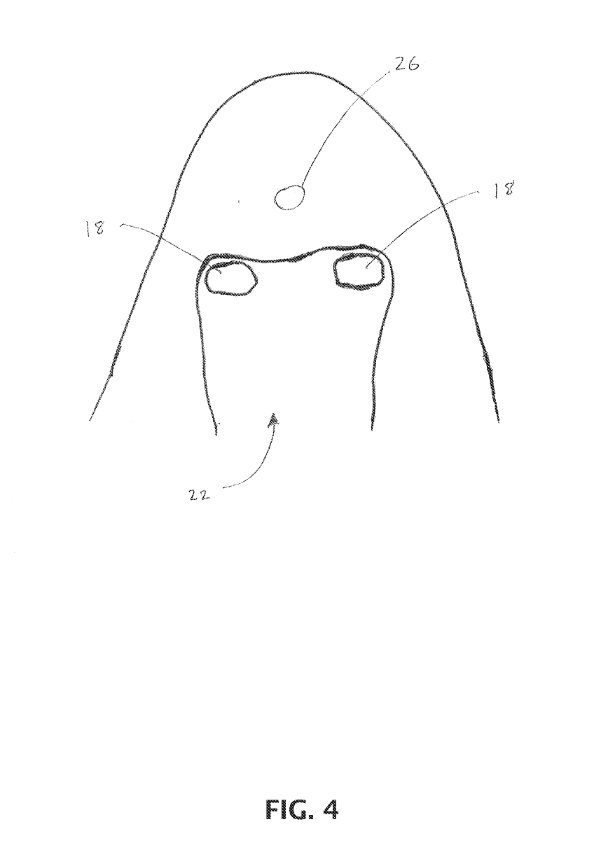

In this embodiment, tip 14 is configured to penetrate tissue (e.g., bladder neck, vaginal, etc.) of a patient at a target area 18. Target area 18 can be, for example, within a patient's vagina 22 and/or near a patient's urethra 26 (e.g., as depicted in FIGS. 3 and 4). In the embodiment shown, tip 14 is configured to penetrate tissue at target area 18 (e.g., tissue within vagina 22 and/or near urethra 26) without the tissue having been incised prior to penetration of the tip (e.g., separately from the insertion of tip 14 or with a tool that is separate from tip 14). In other embodiments, target area 18 can be incised (e.g., with a scalpel) prior to implanting at least one fixation device 10. In the embodiment shown, fixation device 10 is configured to function without implanting tip 14 into a bone. Thus, for example, fixation device 10 can be configured to be deployed and/or implanted into soft tissue (e.g., soft tissue near urethra 22 and/or a bladder neck) so as to secure and/or stabilize the soft tissue and/or surrounding tissues (e.g., urethra 22, a bladder neck, and/or surrounding tissues). In the embodiment shown, tip 14 can comprise any suitable biocompatible material (e.g., silicone, thermoplastic elastomers, polyvinyl chloride, fluoropolymers, polyurethane, polycarbonate, acrylic compounds, thermoplastic polyesters, polypropylene, low density polyethylene, nylon, sulfone resins, high density polyethylene, natural polymers, biocompatible ceramics, biocompatible metals, and the like, and/or combinations thereof) configured (e.g., with a sharpened distal edge and/or a rigidity sufficiently great to enable tip 14) to penetrate tissue at a target area 18 without the need to first separately incise the tissue to properly position the fixation device for tissue suspension. In other embodiments, for example, if target area 18 is first incised, tip 14 may not be configured to penetrate tissue without first incising the tissue (e.g., tip 14 may not have a sharp distal point and/or edge, and/or may be less rigid). In some embodiments, tip 14 comprises one or more suturing material(s) (e.g., nylon, polypropylene, prolene, silk, polyvinylidene fluoride, polyester, stainless steel, titanium, and the like) or combination thereof.

In the embodiment shown, fixation device 10 further comprises a non-absorbable suture 30 having first end 34 coupled to tip 14 and second end 38. Non-absorbable suture 30 can be coupled to tip 14, for example, by looping, knotting, tying, and/or the like. Tip 14 can comprise at least one extension 39 (e.g., two extensions 39, in the embodiment shown) configured to permit non-absorbable suture 30 to be coupled to tip 14. Extensions 39 can be coupled to or unitary with any suitable portion of the tip, such as lower portion 16, as shown in FIG. 5. The coupling may be achieved in any suitable fashion (e.g., via welding, an adhesive, or the like); the two may be unitary by, for example, forming them from a single piece of material. The configuration of at least one extension 39 permits non-absorbable suture 30 to be coupled to tip 14 by opening 40, as shown in FIG. 5, which is completely bounded by extension 39 (which has a handle-like shape) and lower portion 16. In other embodiments, a completely bounded opening need not be used, and such a configuration may be achieved by shaping at least one extension 39 with a slot in which a portion of the suture would reside that would inhibit the suture from being de-coupled from the tip during use. Each of extensions 39 comprises opening 40 configured such that non-absorbable suture 30 can be looped, knotted, tied, or otherwise secured through opening 40. In other embodiments, non-absorbable suture 30 can be coupled to tip 14 with one or more biocompatible adhesive(s). In other embodiments, tip 14 and non-absorbable suture 30 are unitary (i.e., are both formed of a single piece of material). Tip 14 and non-absorbable suture 30 can comprise the same or different materials. Non-absorbable suture 30 can comprise any suitable non-absorbable suture material, such as, nylon, polypropylene, polyamide, prolene, silk, polyvinylidene fluoride, polyester, stainless steel, titanium, and the like, and/or combinations thereof. In the embodiment shown, non-absorbable suture 30 can comprise a single strand of suture, dual strands of suture, or more (e.g., three or more strands of suture). In some embodiments, a strand of non-absorbable suture 30 can have a diameter of between 0.1 millimeters and 1 millimeter. In other embodiments, a strand of non-absorbable suture 30 can have a diameter between approximately 0.2 millimeters and 0.5 millimeters. In other embodiments, a strand of non-absorbable suture 30 can have a diameter of greater than 1 millimeter or less than 0.1 millimeter.

In the embodiment shown, non-absorbable suture 30 comprises a plurality of barbs 42 configured to resist movement of fixation device 10 within the tissue of target area 18 (e.g., to prevent fixation device 10 from exiting target area 18). In the embodiment shown, barbs 42 comprise the same or substantially similar material as non-absorbable suture 30 (e.g., are unitary with suture 30). In other embodiments, barbs 42 can comprise a different material. Barbs 42 can be arranged and/or oriented in any manner configured to improve resistance of movement of fixation device 10 within the tissue of target area 18 (e.g., angled at a particular angle from horizontal, oriented longitudinally with respect to tip 14, spaced with a greater or lesser concentration, etc.). In some embodiments, barbs 42 can have a length of approximately 0.01 millimeters to 1 millimeter. In other embodiments, barbs 42 can have a length of approximately 0.03 millimeters to 0.3 millimeters. In other embodiments, barbs 42 can have a length of approximately 0.05 millimeters to 0.5 millimeters. In some embodiments, barbs 42 can have a length of less than 0.05 millimeters or greater than 1 millimeter. Barbs 42 can vary in length along the same non-absorbable suture 30.

In the embodiment shown, fixation device 10 further comprises absorbable suture 46 coupled to second end 38 of non-absorbable suture 30. Absorbable suture 46 can be coupled to non-absorbable suture 30, for example, by looping, knotting, tying and/or the like. In other embodiments, absorbable suture 46 can be coupled to non-absorbable suture 30 by any suitable biocompatible adhesives. In other embodiments, absorbable suture 46 and non-absorbable suture 30 are unitary (e.g., are manufactured such that non-absorbable suture 30 and absorbable suture 46 comprise the same strand or strands of suture). Absorbable suture 46 can comprise any suitable absorbable suturing material, such as, for example, polyglycolide, polyactic acid, polydioxanone, caprolactone, poliglecaprone, plain catgut, chromic catgut, and the like, or combinations thereof. In the embodiment shown, absorbable suture 46 can comprise a single strand of suturing material or more than one strands (e.g., dual strands or more) of suturing material. In the embodiment shown, absorbable suture 46 does not comprise a plurality of barbs. In other embodiments, absorbable suture 46 can comprise a plurality of barbs configured to resist movement of fixation device 10 within the tissue of target area 18 (e.g., to prevent fixation device 10 from exiting target area 18). Barbs 42 can comprise the same, substantially similar, or a different material as absorbable suture 46 (e.g., are unitary with suture 46). Barbs 42 can be arranged, oriented, and/or otherwise configured to resist movement of fixation device 10 within the tissue of target area 18 (e.g., angled at a particular angle from horizontal, oriented longitudinally with respect to tip 14, spaced with greater or lesser concentration, etc.)

In the embodiment shown, fixation device 10 further comprises retainer or stay tab 50 coupled to absorbable suture 46. In the embodiment shown, stay tab 50 comprises at least one opening 51 (e.g., three openings 51, in the embodiment shown). Stay tab 50 is configured to substantially prevent at least some tissue from moving away from tip 14 if fixation device 10 is implanted in the tissue at target area 18. For example, in some embodiments, stay tab 50 is configured to substantially fix the position of tissue relative to second end 38, such as to suspend the neck of a patient's bladder and/or a patient's urethra following implantation of fixation device, such as is depicted in FIGS. 3 and 4. Stay tab 50 can also be configured to limit the depth to which at least a portion of fixation device 10 can enter the tissue of target area 18 (e.g., such that the stay tab will contact a surface of the tissue to prevent fixation device 10 from entering target area 18 at a depth greater than length L of fixation device 10). Stay tab 50 can be coupled to absorbable suture 46, for example, by looping, knotting, tying, and/or the like (e.g., through openings 51). For example, each of openings 51 can have a diameter of 0.05 millimeters to 1 centimeter (e.g., 1.5 millimeters), and absorbable suture 46 can be passed through one or more of openings 51 to be looped, knotted, tied, and/or otherwise secured. In other embodiments, stay tab 50 can be coupled to absorbable suture 46 by any suitable biocompatible adhesives. In other embodiments, stay tab 50 and absorbable suture 46 are unitary (i.e., are both formed of a single piece of material). Stay tab 50 can comprise any suitable biocompatible material (e.g., silicone, thermoplastic elastomers, polyvinyl chloride, fluoropolymers, polyurethane, polycarbonate, acrylic compounds, thermoplastic polyesters, polypropylene, low density polyethylenes, nylon, sulfone resins, high density polyethylenes, natural polymers, biocompatible ceramics, biocompatible metals, and the like) and/or combinations thereof. In the embodiment shown, absorbable suture 46 is configured to dissolve and/or be absorbed by a patient's body such that stay tab 50 decouples from fixation device 10 (e.g., such that stay tab 50 is released from target area 18 and can exit a patient, for example, by extrusion from the vagina). Absorbable suture can be configured to absorb (e.g., such that stay tab 50 decouples from fixation device 10) after at least two weeks (e.g., two, three, four weeks, or more).

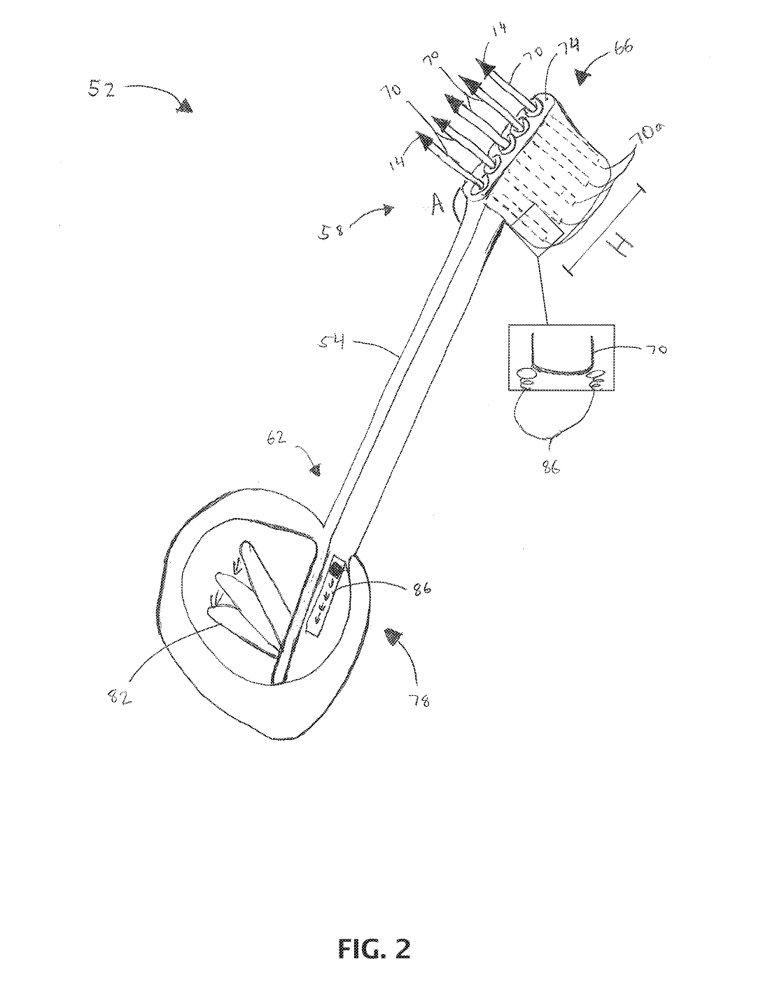

FIG. 2 depicts an embodiment of an apparatus 52 that is configured to deploy one or more of the present fixation devices (e.g., fixation devices 10). In the embodiment shown, apparatus 52 comprises shaft 54 having first end 58 and second end 62. Shaft 54 can comprise any material that can be sterilized for use with a patient. For example, shaft 54 can comprise a biocompatible material, such as, silicone, thermoplastic elastomers, polyvinyl chloride, fluoropolymers, polyurethane, polycarbonate, acrylic compounds, thermoplastic polyesters, polypropylene, low density polyethylene, nylon, sulfone resins, high density polyethylene, natural polymers, biocompatible ceramics, biocompatible metals (e.g., stainless steel, titanium, etc.), and the like, or combinations thereof.

In the embodiment shown, head 66 is coupled to first end 58 of shaft 54 (e.g., at an angle A, as shown). Angle A can, for example, be between 150 and 180 degrees (e.g., equal to or between any two of: 150, 155, 160, 165, 170, 175, and/or 180 degrees). In other embodiments, angle A can be any suitable angle configured to permit head 66 to be positioned substantially parallel to the tissue at a target area (e.g., target area 18). In the embodiment shown, head 66 has a length H that is between 0.5 centimeters and 2 centimeters. In other embodiments, length H can be any suitable length configured to correspond to a given target area (e.g., target area 18). Head 66 can comprise the same material or a different material than shaft 54, such as, for example, any of the materials discussed above for shaft 54.

In the embodiment shown, head 66 comprises plurality of guides 70. In this embodiment, guides 70 are disposed within head 66 (e.g., partially or entirely within head 66) when in a retracted position as shown. In the embodiment shown, head 66 comprises five guides 70. In other embodiments, head 66 can comprise less than five guides 70 (e.g., four, three, or less) or more than five guides 70 (e.g., six, seven, or more). In the embodiment shown, guides 70 are disposed in one row. In other embodiments, guides 70 can be disposed in any arrangement (e.g., two rows, three rows, a zigzag, a diamond, a square, etc.) configured to correspond to a target area (e.g., target area 18) and/or configured to assist in deployment of a fixation device (e.g., fixation device 10) in a given target area (e.g., target area 18). Guides 70 can comprise any suitable length, such as, for example, a length of 0.1 millimeter to 1 centimeter (e.g., 3 millimeters). Guides 70 can also comprise any suitable diameter, such as, for example, a diameter of 0.1 millimeter to 1 centimeter (e.g., 3 millimeters).

In the embodiment shown, each guide 70 is (i) configured to at least partially accommodate a fixation device (e.g., fixation device 10), (ii) configured to extend from head 66 into a deployed configuration, (iii) configured to retract into head 66 into a retracted configuration, and (iv) biased toward the retracted configuration (e.g., as indicated by hidden lines 70a). In some embodiments, each guide 70 can be configured to accommodate more than one fixation device (e.g., two, three, or more fixation devices 10). In other embodiments, guides 70 can be biased toward the deployed configuration. In other embodiments, guides 70 are not biased toward either the deployed or the retracted configuration. Guides 70 can comprise any material that can be sterilized for use with a patient (e.g. stainless steel, titanium, plastic, etc.) of a patient at a target area (e.g., target area 18). Guides 70 can further be configured to assist in penetrating a target area (e.g., by comprising a serrated edge, etc.). In some embodiments, apparatus 52 can be configured to allow for separate and/or individualized deployment of fixation device(s) 10. In other embodiments, apparatus 52 can allow for concurrent deployment of all fixation devices 10.

In the embodiment shown, a plurality of fixation devices (e.g., plurality of fixation devices 10) can be disposed within a corresponding guide 70. In the embodiment shown, head 66 comprises a distal surface 74, and apparatus 52 and/or fixation device (e.g., fixation device 10) can be configured such that a fixation device 10 can be partially disposed within a corresponding guide 70 such that tip 14 of the fixation device extends from head 66 beyond surface 74 when the guide is in the retracted configuration). In some embodiments, a fixation device (e.g., fixation device 10) can be disposed within head 66 such that distal surface 74 defines an outer perimeter of apparatus 52 and the fixation device(s) when guides 70 are in the retracted configuration (e.g., such that substantially no portion of the fixation device(s) extends beyond surface 74). In the embodiment shown, guides 70 can be cylindrical and/or tubular with a transverse dimension of less than a transverse dimension of tip 14 such that if guides 70 extend from head 66, guides 70 can press against tip 14 (e.g., against bottom surface 76 of tip 14, as shown in FIGS. 5-8) to extend fixation device 10 from head 66 into tissue at a target area (e.g., target area 18). In such embodiments, guides 70 can have a transverse dimension (e.g., inner diameter) that is less than a transverse dimension of barbs 42 when the barbs are in a relaxed configuration, such that guides 70 compresses or maintains the barbs in a compressed configuration until the guide is withdrawn and the barbs are allowed to expand.

In other embodiments, guides 70 can comprise a transverse dimension substantially similar to the transverse dimension of tip 14 such that guides 70 partially compress tip 14 (e.g., such that tip 14 extends with guides 70 into tissue of a target area). Tip 14 can then be decompressed (e.g., released) by a switch (discussed in detail below). In other embodiments, guides 70 can comprise a transverse dimension greater than transverse dimension of tip 14 (e.g., such that tip 14 can be disposed entirely within guides 70). In this embodiment, guides 70 can comprise an inner piston and/or the like configured to extend fixation device 10 from guides 70. In the embodiment shown, stay tab 50 can comprise a transverse dimension of less than the transverse dimension of guides 70 such that stay tab 50 can be disposed within a guide without bending and/or compressing and/or angling stay tab 50. In other embodiments, stay tab 50 can comprise a transverse dimension of substantially equal to or greater than the transverse dimension of guides 50. In this embodiment, stay tab 50 can be compressed and/or bent and/or angled to accommodate stay tab 50 within guides 70. In the embodiment shown, apparatus 52 is configured such that at least one guide 70 enters a target area (e.g., target area 18) along with at least one fixation device (e.g., fixation device 10). Apparatus 52 is further configured such that after insertion of a fixation device (e.g., fixation device 10) the corresponding guide 70 retracts from the target area (e.g., target area 18) into the retracted configuration, and the inserted fixation device remains within the tissue at the target area.

In the embodiment shown, apparatus 52 further comprises handle 78 extending from second end 62 of shaft 54. In some embodiments, handle 78 can be coupled to second end 62 of shaft 54 (e.g., unitary with shaft 54). In some embodiments, handle 78 can be configured to accommodate a shape and/or contour of (e.g., to be received in) a user's hand. In the embodiment shown, handle 78 comprises lever 82 configured to be actuated (e.g., depressed and/or engaged by a user) to extend at least one of plurality of guides 70 from head 66 (e.g., into the deployed configuration). For example, when head 66 is disposed at a target area (e.g., target area 18), lever 82 can be actuated such that at least one of the plurality of guides 70 and one or more corresponding fixation devices (e.g., fixation devices 10) can enter (e.g., penetrate) tissue of a patient at a target area (e.g., target area 18). In some embodiments, lever 82 can be configured to deploy all of guides 70 with one actuation (e.g., depression) of the lever. In other embodiments, lever 82 can be configured to deploy any one of the plurality of guides, or more than one of the plurality of guides, with one actuation. For example, in some embodiments, apparatus 52 is configured to sequentially deploy the fixation members one at time (e.g., to deploy a different one of the fixation devices with each actuation of the lever).

Apparatus 52 can include any suitable force-applying mechanism configured to extend guides 70 from head 66. For example, head 66 can comprise at least one spring 86 (e.g., one, two, or more), where each spring 86 is (i) in a compressed configuration when plurality of guides 70 are in the retracted configuration, (ii) configured to apply a force to at least one of plurality of guides 70 such that at least one guide 70 extends from head 66 into the deployed configuration, (iii) coupled to lever 82 such that lever 82 will release at least one spring 86 from the compressed configuration if lever 82 is actuated, and (iv) configured to stop applying the force to the corresponding guide(s) 70 when the corresponding guide(s) is/are in the deployed configuration (e.g., to permit the corresponding guide(s) 70 to retract into the retracted configuration). Spring 86 can be, for example, a compression spring, a leaf spring, a coil spring, a torsion spring, and the like. In other embodiments, at least one spring 86 can be configured to apply a force to at least one guide 70 in the expanded configuration. Actuating lever 82, for example, can release at least one guide 70 such that relaxation or expansion of spring 86 applies a force to extend at least one guide 70 from head 66. In other embodiments, lever 82 can be coupled to one or more lift(s) or secondary lever(s) beneath plurality of guides 70 such that when lever 82 is actuated, each lift or secondary lever extends one or more of the plurality of guides 70 from head 66. One of ordinary skill in the art will recognize numerous other force-applying mechanisms to permit plurality of guides 70 to extend from head 66.

In the embodiment shown, handle 78 further comprises switch 86 configured to release at least one of a plurality of fixation devices (e.g., plurality of fixation devices 10) from at least one of plurality of guides 70 when the at least one fixation device(s) (e.g., fixation devices 10) is within the tissue at a target area (e.g., target area 18) and/or when the respective guide(s) is in the extended position. In the embodiment shown, switch 86 can be configured to release at least one of tip 14, non-absorbable suture 30, absorbable suture 46, and/or stay tab 50. In the embodiment shown, switch 86 can be configured to electrically and/or mechanically release one or more portions of a fixation device (e.g., fixation device 10). For example, switch 86 can retract a retainer (e.g., extending though absorbable suture 46 and/or non-absorbable suture 30) to release the respective fixation device(s). In other embodiments, guides 70 can be configured to compress tip 14 to permit tip 14 to extend with guides 70. Switch 86 can be configured to relax guides 70 such that tip 14 is no longer compressed (i.e., releasing fixation device(s) 10 within a target area). In other embodiments, switch 86 can release a grip and/or retainer acting on stay tab 50 to release the respective fixation device(s).

The present invention also contemplates one or more kits, including, but not limited to, one or more of apparatus 52 (or components thereof) and/or one or more of fixation device(s) 10 (or components thereof). In some embodiments, a kit can further include instructions and/or methods (e.g., in a hard copy and/or computer readable) for how to use apparatus 52 and/or fixation device(s) 10.

Embodiments of the present methods include suspending tissue (e.g., during a urethropexy) of a patient at a target area (e.g., target area 18, such as, for example, a bladder neck). For example, such methods can comprise (i) inserting into a target area (e.g., a bladder neck within vagina 22) of a patient an apparatus (e.g., apparatus 52) comprising a plurality of fixation devices (e.g., fixation devices 10) disposed at least partially within a plurality of guides (e.g., plurality of guides 70), (ii) positioning a portion of the apparatus (e.g., apparatus 52) substantially parallel to tissue of the patient at a target area (e.g. near urethra 26), (iii) deploying the apparatus (e.g., apparatus 52) such that at least one of the plurality of guides (e.g., at least one of guides 70) and at least one of the corresponding plurality of fixation devices (e.g., at least one of fixation devices 10) penetrates the tissue at the target area (e.g., near urethra 26), (iv) releasing the at least one fixation device (e.g., fixation device 10) at least partially within the tissue at the target area (e.g., target area 18) with a switch (e.g., switch 86), (v) allowing the at least one guide (e.g., at least one of guides 70) to retract into a retracted configuration, and (vi) repeating steps (iii)-(v) until a sufficient number of the plurality of fixation devices (e.g., fixation devices 10) are at least partially within the tissue at a target area (e.g., target area 18) to suspend the tissue (e.g., such that urethra 26 can be suspended). The method can further comprise (vii) permitting an absorbable suture (e.g., absorbable suture 46 of fixation device 10) to be absorbed such that at least one stay tab (e.g., stay tab 50 of fixation device 10) can decouple from the fixation device (e.g., such that stay tab 50 can be permitted to exit a patient). For example, if the method is used in a urethropexy, the method can comprise deploying the apparatus (e.g., apparatus 52) on either side of urethra 26 (e.g., by successively actuating lever 82 of handle 78). Alternatively, an apparatus (e.g., apparatus 52) can positioned such that at least one of the plurality of guides (e.g., at least one of plurality of guides 70) is positioned on either side of urethra 26 such that the apparatus (e.g., apparatus 52) can deploy at least one of a plurality of fixation devices (e.g. fixation devices 10) on either side of urethra 26 without repositioning the apparatus (e.g., without having to move apparatus 52 laterally or longitudinally with respect to target areas 18).

Embodiments of the present kits can comprise at least one of the present apparatuses (e.g., apparatus 52) disposed in a sealed and/or sterile container (e.g., a sealed plastic bag). Some embodiments further comprises at least one of the present fixation devices (e.g., device 10). Some embodiments further comprise instructions for using the apparatus and/or the fixation device (e.g., in accordance with one or more embodiments of the present methods).

The above specification and examples provide a complete description of the devices, apparatuses, and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling within the scope of the claims, and embodiments other than the one shown may include some or all of the features of the depicted embodiment. For example, components may be combined as a unitary structure and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A fixation device comprising:
a tip configured to penetrate tissue of a patient at a target area;
a non-absorbable suture having a first end coupled to the tip and a second end, the non-absorbable suture comprising a plurality of barbs configured to resist movement of the fixation device within the tissue of the target area;
an absorbable suture coupled to the second end of the non-absorbable suture; and
a stay tab coupled to the absorbable suture, the stay tab configured to substantially prevent at least some tissue from moving away from the tip if the fixation device is implanted in the tissue of the target area.

2. The fixation device of claim 1, where the stay tab is further configured to limit the depth to which at least a portion of the fixation device can enter the tissue of the target area.

3. The fixation device of claim 1, where the tip is configured to penetrate the tissue at the target area without the tissue having been incised prior to penetration of the tip.

4. The fixation device of claim 1, where the tip comprises a first material, the non-absorbable suture comprises a second material, the absorbable suture comprises a third material, and the stay tab comprises a fourth material.

5. The fixation device of claim 4, where the first material is the same as the second material.

6. The fixation device of claim 4, where the first material is silicone.

7. The fixation device of claim 4, where the first material is a biocompatible material configured with sufficient rigidity to penetrate tissue at the target area without the tissue having been incised prior to penetration of the tip.

8. The fixation device of claim 1, where the absorbable suture comprises a plurality of barbs configured to resist movement of the fixation device within the tissue of the target area.

9. (canceled)

10. The fixation device of claim 1, where the absorbable suture is configured to be absorbed such that the stay tab will not de-couple from the non-absorbable suture in less than two weeks.

11. The fixation device of claim 1, where the absorbable suture is configured to be absorbed such that the stay tab will de-couple from the non-absorbable suture in two to four weeks.

12-23. (canceled)

24. An apparatus to deploy a fixation device comprising:
a shaft having a first end and a second end;
a head coupled to the first end of the shaft, the head comprising a plurality of guides disposed within the head, where each of the plurality of guides is:
(i) configured to at least partially accommodate a fixation device;
(ii) configured to extend from the head into a deployed configuration;
(iii) configured to retract into the head into a retracted configuration; and
(iv) biased toward the retracted configuration; and
a handle extending from the second end of the shaft, the handle comprising a lever configured to be actuated to extend at least one of the plurality of guides from the head.

25. The apparatus of claim 24, where a plurality of fixation devices are at least partially disposed within the plurality of guides.

26. The apparatus of claim 25, where the plurality of fixation devices comprise:
a tip configured to penetrate tissue of a patient at a target area;
a non-absorbable suture having a first end coupled to the tip and a second end, the non-absorbable suture comprising a plurality of barbs configured to resist movement of the fixation device within the tissue of the target area;
an absorbable suture coupled to the second end of the non-absorbable suture; and
a stay tab coupled to absorbable suture, the stay tab configured to substantially prevent at least some tissue from moving away from the tip if the fixation device is implanted in the tissue of the target area.

27. (canceled)

28. The apparatus of claim 26, where the plurality of fixation devices are disposed within the head such that a surface of the head from which the plurality of guides extends is substantially smooth when the plurality of guides are in the retracted configuration.

29-35. (canceled)

36. The apparatus of claim 24, where the head is coupled to the first end of the shaft at an angle between 150 degrees and 180 degrees.

37-41. (canceled)

42. A method of suspending tissue of a patient at a target area comprising:
(i) inserting into a vagina of a patient an apparatus comprising a plurality of fixation devices disposed at least partially within a plurality of guides; and
(ii) deploying the apparatus such that at least one fixation device penetrates the tissue at the target area without the tissue having been incised prior to penetration of the at least one fixation device.

43-46. (canceled)

47. The method of any of claim 42, where the apparatus comprises:
a shaft having a first end and a second end;
a head coupled to the first end of the shaft, the plurality of guides disposed within the head, where each of the plurality of guides is:
(i) configured to at least partially accommodate at least one of the plurality of fixation device;
(ii) configured to extend from the head into a deployed configuration;
(iii) configured to retract into the head into the retracted configuration; and
(iv) biased toward the retracted configuration,
a handle extending from the second end of the shaft, the handle comprising a lever configured to be actuated to extend at least one of the plurality of guides from the head into the deployed configuration.

48. The method of claim 47, where the plurality of fixation devices each comprises:
a tip configured to penetrate tissue of a patient at the target area;
a non-absorbable suture having a first end coupled to the tip and a second end, the non-absorbable suture comprising a plurality of barbs configured to resist movement of the fixation device within the tissue at the target area;
an absorbable suture coupled to the second end of the non-absorbable suture; and
a stay tab coupled to the absorbable suture, the stay tab configured to substantially prevent at least some tissue from moving away from the tip if the fixation device is implanted in the tissue at the target area.

49. (canceled)

50. The method of claim 48, where the apparatus is positioned such that at least one of the plurality of guides is positioned on either side of the urethra such that the apparatus can deploy at least one of a plurality of fixation devices on either side of the urethra without repositioning the apparatus.

51-55. (canceled)

56. The method of claim 42, where the plurality of fixation devices each comprises:
a tip configured to penetrate tissue of a patient at the target area;
a non-absorbable suture having a first end coupled to the tip and a second end, the non-absorbable suture comprising a plurality of barbs configured to resist movement of the fixation device within the tissue at the target area;
an absorbable suture coupled to the second end of the non-absorbable suture; and
a stay tab coupled to the absorbable suture, the stay tab configured to substantially prevent at least some tissue from moving away from the tip if the fixation device is implanted in the tissue at the target area.

57. An apparatus to deploy a fixation device comprising:
a shaft having a first end and a second end;
a head coupled to the first end of the shaft, and a guide disposed within the head, where the guide is:
(i) configured to at least partially accommodate a fixation device, where the fixation device comprises:
a first portion configured to penetrate tissue of a patient without the tissue having been incised;
a stay tab configured to substantially prevent at least some tissue from moving away from the first portion; and
a suture coupling the first portion to the stay tab;
(ii) configured to extend from the head into a deployed configuration; and
(iii) configured to retract into the head into a retracted configuration; and
a handle extending from the second end of the shaft, the handle comprising a lever configured to be actuated to extend the guide from the head.
